(19) United States
(12) Patent Application Publication
(10) Pub. No.: US 2017/0165461 A1
(43) Pub. Date: Jun. 15, 2017

(54) TREATING AN OBJECT WITH A GASEOUS COMPOUND IN AN AIRTIGHT SPACE

(71) Applicant: Novomic LTD., Rosh HaAyin (IL)

(72) Inventor: Zvi Yemini, Tel Aviv-Yaffo (IL)

(21) Appl. No.: 15/438,842

(22) Filed: Feb. 22, 2017

Related U.S. Application Data
(63) Continuation-in-part of application No. 15/092,631, filed on Apr. 7, 2016.
Application No. 15/092,631 is a continuation of application No. 13/544,269, filed on Jul. 9, 2012, now Pat. No. 9,307,820.
Application No. 13/544,269 is a continuation-in-part of application No. 12/901,544, filed on Oct. 10, 2010, now abandoned.
Application No. 12/901,544 is a continuation-in-part of application No. 12/473,058, filed on May 27, 2009, now abandoned.
Application No. 12/473,058 is a continuation-in-part of application No. PCT/IL2008/000031, filed on Jan. 6, 2008.
(60) Provisional application No. 60/878,351, filed on Jan. 4, 2007.

(51) Int. Cl. A61M 35/00; A01N 25/08; A01N 25/18; A01N 37/02

(52) U.S. Cl. CPC A61M 35/00; A01N 37/02; A01N 25/08; A01N 25/18; A61M 2202/02

(57) ABSTRACT

A system, capsule, and method for storage and delivery of an active agent for treatment of an object.

### CROSS-REFERENCE TO RELATED APPLICATIONS

The present application is a continuation-in-part of U.S. patent application Ser. No. 15/092,631 filed Apr. 7, 2016, which is a continuation of U.S. patent application Ser. No. 13/544,269 filed Jul. 9, 2012, which is a continuation-in-part of U.S. patent application Ser. No. 12/901,544 filed on Oct. 10, 2010 which is a continuation-in-part of U.S. patent application Ser. No. 12/473,058 filed on May 27, 2009, which is a continuation-in-part of International Patent Application No. PCT/IL2008/000031 filed Jan. 6, 2008, which claims priority from U.S. Provisional Patent Application No. 60/878,351, filed Jan. 4, 2007, all of which are incorporated herein by reference as if fully set forth herein.

### FIELD AND BACKGROUND OF THE INVENTION

The present invention relates to the field of medical treatment systems, and more particularly, to a system and a method for treating a subject with a gaseous compound in an airtight space, such as treatment of lice and nits within an airtight cap.

Infestation of the human body by lice is an increasingly prevalent social and health problem in many countries in the world. Lice infest hundreds of millions of people each year. Lice are very small insects, about 2-3 mm in length. They deposit eggs either on a hair or fabric fiber and attach them firmly with a cement-like excretion. The life cycle of lice comprises an egg stage, three nymphal stages, and an adult stage, and takes about one month. The nymphs, (the larval stage in insect development), and adults suck blood, causing disturbance (itching, also known as pruritus) and secondary infection.

Lice, being insects, use tracheae for respiration. Tracheae are a system of internal tubes (invaginations of the cuticle) penetrating the insect's body, through which air diffuses or is being pumped directly to the body tissues. Within body cells, chemical respiration takes place in the mitochondria, where ATP is produced under oxygen consumption. The louse's hard chitinous exoskeleton serves as protection from external elements. Lice eggs (or ova) are similarly protected by a chitinous sheath surrounding the eggs and attached to the hair shaft.

Although lice may be affected by the use of an insecticide, the eggs often remain resistant to attack. Thus, present art optimum treatment of a lice infestation includes both a pediculicide, which kills the adult lice, and an ovicide, which interrupts the development of the eggs.

Treatment for eliminating head lice traditionally included home remedies such as smearing mayonnaise, olive oil, hair pomade, or some other heavily viscous material about an infested scalp coupled with rigorous combing of the hair and meticulous removal of adult lice, nymphs, and nits. Though these home remedies do not kill head lice, the prevailing thought is that the viscosity of the material makes it hard for head lice to roam about the scalp, making for easy removal. Such home remedies are usually ineffective at controlling head lice due to the ability of the lice to revive rapidly once these materials are removed.

More effective treatments for eliminating head lice involve massaging the infested scalp with over-the-counter (OTC) topical creams, liquids, or lotions, containing active insecticides. Because of their potential toxicity to the human host, the use of these topical formulas is regulated by the FDA. Over-the-counter insecticides typically include pyrethrins or permethrin as the active ingredients.

Biologically active agents have been used for some time in attempts to control lice. For example, lindane (gammabenzene hexachloride), organophosphates (malathion), natural pyrethrins, and synthetic compounds known as pyrethroids (such as permethrin) have been used as pediculicides in lice treatment formulations. These agents however, have drawbacks. Lindane has a poor safety profile, and lice have developed resistance to it. Natural pyrethrin requires frequent follow-up treatments because it provides only short term residual action. Synthetic pyrethroids, although more effective against lice than natural pediculicides, are often more toxic to the subject being treated.

Additionally, strains of head lice have been identified worldwide which are resistant to all currently available topical treatments. Possible neural damage to the human host prevents raising the insecticide levels above the current threshold in an attempt to combat these newer treatment resistant head lice.

As such, there is a need for a system for treatment of lice which is reliable in killing the lice and eggs, and which does not require use of potentially harmful active ingredients.

### SUMMARY OF THE INVENTION

Some embodiments of the invention relate to medical treatment systems, and more particularly, to a system and a method for treating a subject with a gaseous compound in an airtight space, such as treatment of lice and nits within an airtight cap.

According to an aspect of some embodiments of the invention, there is provided a capsule for storage and delivery of an active agent, the capsule including:

a container defining a storage volume, the storage volume containing a substrate having a volume of liquid active agent adsorbed onto the substrate or absorbed in the substrate;

at least one fluid flow passage into and out of the storage volume; and

at least one seal sealing the at least one fluid flow passage and preventing flow of fluid therefrom,

wherein the capsule is sealed to fluid flow out of the capsule.

In some embodiments, the container is formed of a fluid impassable material.

In some embodiments, the substrate includes a plurality of unbound miniscule particles.

In some embodiments, the capsule further includes at least one cover adapted to be disposed about the at least one seal, wherein the at least one cover includes:

a puncturing element adapted to puncture the seal; and

at least one channel,

wherein, in an actuated state of the capsule, puncturing element punctures the seal such that the at least one channel is in fluid flow communication with the at least one fluid flow passage, and fluid can flow into and out of the storage volume via the at least one fluid flow passage and via the at least one channel

In some embodiments, in the actuated state of the capsule, the cover is positioned axially closer to the storage volume than in a storage state of the capsule, thereby causing the puncturing element to puncture the seal.

In some embodiments, the capsule includes two the seals sealing the storage volume, the capsule includes two the covers, each including a the puncturing element and a the channel, each puncturing element being aligned with a corresponding one of the two seals, and in the actuated state, each of the puncturing elements punctures a the seal corresponding thereto, thereby to allow flow of fluid in the corresponding channel.

In some embodiments, in the actuated state, a first of the two channels functions as an inlet and a second of the two channels functions as an outlet.

In some embodiments, the two seals and the two corresponding covers are disposed axially on opposite sides of the storage volume.

In some embodiments, the capsule further includes at least one filter disposed between the storage volume and the fluid flow passage, and enabling filtering of fluid flowing through the fluid flow passage following removal or breakage of the seal.

In some embodiments, the at least one filter prevents passage of the substrate and allows passage of a vapor of the liquid active agent.

According to another aspect of some embodiments of the invention, there is provided a system for evaporating and circulating an active agent disposed in a capsule, the system including:

the capsule as described hereinabove;

a vaporizing and circulating gas source in fluid flow communication with the storage volume via the at least one channel,

the gas source adapted to emit a vaporizing a circulating gas into the storage volume thereby to increase pressure within the storage volume, the increase in pressure causing vaporization of the active agent and release thereof from the substrate.

In some embodiments, the vaporizing and circulating gas is selected from the group consisting of: pure oxygen, pure carbon dioxide, and ambient air.

In some embodiments, the capsule includes a first and a second the seal sealing the storage volume, the capsule includes a first and a second the cover, each including a the puncturing element and a the channel, each puncturing element being aligned with a corresponding one of the first and second seals, in the actuated state, each of the puncturing elements punctures a the seal corresponding thereto, thereby to allow flow of fluid out of the storage volume via the corresponding channel, the fluid including a mixture of a vapor of the active agent and the vaporizing and circulating gas, and the gas source is in fluid flow communication with a the channel of the first cover, the channel of the first cover functioning as an inlet into the storage volume.

In some embodiments, the system further includes an object to be treated with the active agent and a fluid-tight enclosure enclosing the object and defining an internal treatment volume, the internal treatment volume being in fluid flow communication with the channel of the second cover, wherein the gas flows from the gas source into the storage volume, increases pressure within the storage volume thereby vaporizing the active agent, and circulates vapor of the active agent, via the channel of the second cover, into the fluid tight enclosure, for treatment of the object.

In some embodiments, the object is a head of a subject.

In some embodiments, the active agent is acetic acid.

Unless otherwise defined, all technical and scientific terms used herein have the same meaning as commonly understood by one of ordinary skill in the art to which the invention pertains. In case of conflict, the specification, including definitions, will take precedence.

As used herein, the terms “comprising”, “including”, “having” and grammatical variants thereof are to be taken as specifying the stated features, integers, steps or components but do not preclude the addition of one or more additional features, integers, steps, components or groups thereof These terms encompass the terms “consisting of” and “consisting essentially of”.

As used herein, the indefinite articles “a” and “an” mean “at least one” or “one or more” unless the context clearly dictates otherwise.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. Throughout the drawings, like-referenced characters are used to designate like elements.

In the drawings:

FIG. 1 is a schematic illustration of a system for treating lice and nits on a head of a user according to an embodiment of the teachings herein;

FIG. 2 is a schematic illustration of a capsule according to an embodiment of the teachings herein, which capsule may form part of the system of FIG. 1;

FIG. 3 is a schematic block diagram of the system of FIG. 1 using the capsule of FIG. 2;

FIG. 4A is a schematic illustration of a cap for treating lice and nits on a head of a user according to an embodiment of the teachings herein, which cap may form part of the system of FIG. 1;

FIG. 4B is a sectional illustration of an injection tube forming part of the cap of FIG. 4A, the sectional illustration taken along section lines IVB-IVB in FIG. 4A;

FIG. 5A is a perspective illustration of an active agent capsule according to another embodiment of the teachings herein, which capsule may form part of the system of FIG. 1;

FIGS. 5B and 5C are, respectively, a side view planar illustration and a top view planar illustration of the active agent capsule of FIG. 5A;

FIG. 5D is a sectional view illustration of the active agent capsule of FIGS. 5A to 5C, the sectional illustration taken along section lines VD-VD in FIG. 5C;

FIG. 5E is a perspective view illustration of a filter compartment and a filter forming part of the active agent capsule of FIGS. 5A to 5D;

FIG. 6A is a perspective partially exploded view illustration of an active agent capsule according to yet another embodiment of the teachings herein, which capsule may form part of the system of FIG. 1;

FIGS. 6B, 6C, and 6D are, respectively, a fully exploded, partially exploded, and assembled view sectional illustrations of the active agent capsule of FIG. 6A;

FIG. 6E is a planar top view illustration of a filter compartment and a filter forming part of the active agent capsule of FIGS. 6A to 6D;

FIG. 7 is a schematic illustration of a porous particle which may be used in an active ingredient capsule, such as the capsules of FIGS. 5A to 5E and 6A to 6F, according to an embodiment of the teachings herein;

FIG. 8 is a schematic illustration of another cap for treating lice and nits on a head of a user according to another embodiment of the teachings herein, which cap may form part of the system of FIG. 1;

FIGS. 9A and 9B are schematic block diagrams of two embodiments of a system for treating an object with gas or vapor according to additional embodiments of the teachings herein; and

FIG. 10 is a flow chart of a method for treating lice and nits on a head of a subject, according to an embodiment of the teachings herein.

### DETAILED DESCRIPTION OF EMBODIMENTS

The invention, in some embodiments, relates to the field of medical treatment systems, and more particularly, to a system and a method for treating a subject with a gaseous compound in an airtight space, such as treatment of lice and nits within an airtight cap.

The principles, uses and implementations of the teachings herein may be better understood with reference to the accompanying description and figures. Upon perusal of the description and figures present herein, one skilled in the art is able to implement the invention without undue effort or experimentation.

Before explaining at least one embodiment of the invention in detail, it is to be understood that the invention is not limited in its applications to the details of construction and the arrangement of the components and/or methods set forth in the following description and/or illustrated in the drawings and/or the Examples. The invention can be implemented with other embodiments and can be practiced or carried out in various ways. It is also understood that the phraseology and terminology employed herein is for descriptive purpose and should not be regarded as limiting.

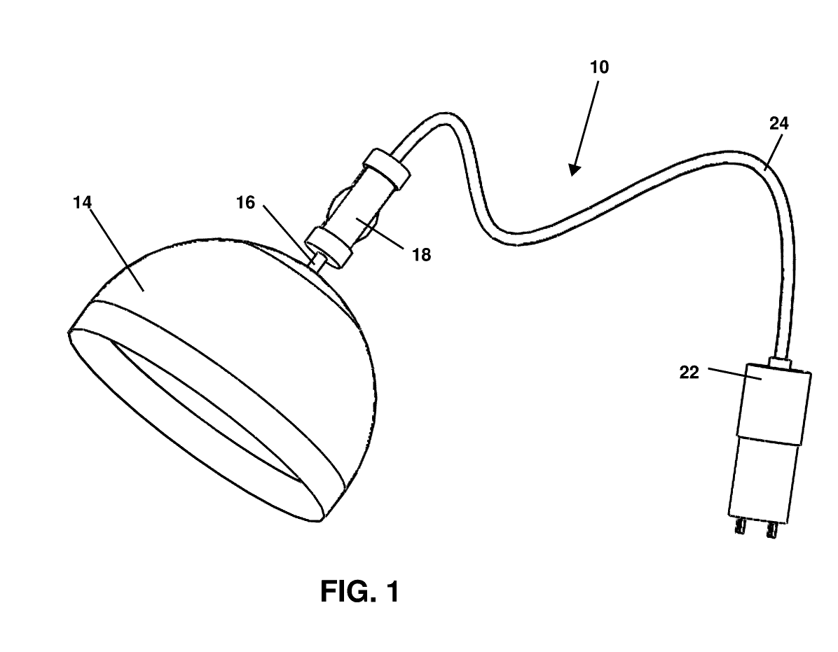

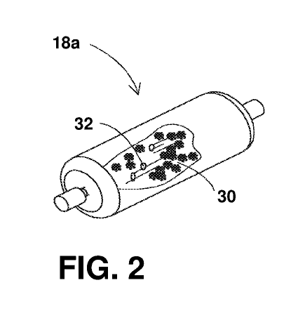

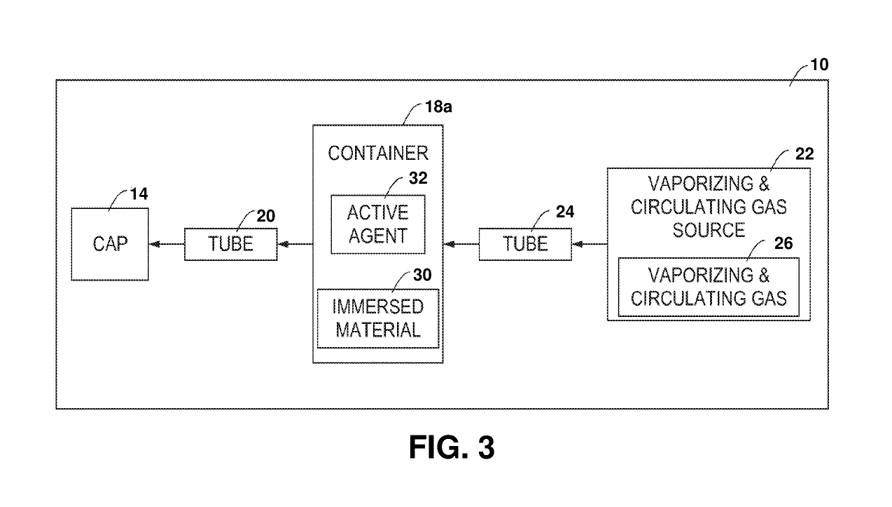

Reference is now made to FIG. 1, which is a schematic illustration of a system for treating lice and nits on a head of a user according to an embodiment of the teachings herein, to FIG. 2, which is a schematic illustration of a capsule which may form part of the system of FIG. 1, according to an embodiment of the teachings herein, and to FIG. 3, which is a schematic block diagram of the system of FIG. 1 using the capsule of FIG. 2. A system similar to that of FIGS. 1 and 3, and the capsule of FIG. 2, as described herein, are also described in U.S. patent application Ser. No. 13/544,269 filed Jul. 9, 2012, U.S. patent application Ser. No. 12/901,544 filed on Oct. 10, 2010, and U.S. patent application Ser. No. 12/473,058 filed on May 27, 2009, of which the present application is a continuation in part, as well as in Applicant's corresponding European Patent Application No. 09163262.0, all of which are hereby incorporated by reference as if fully set forth herein.

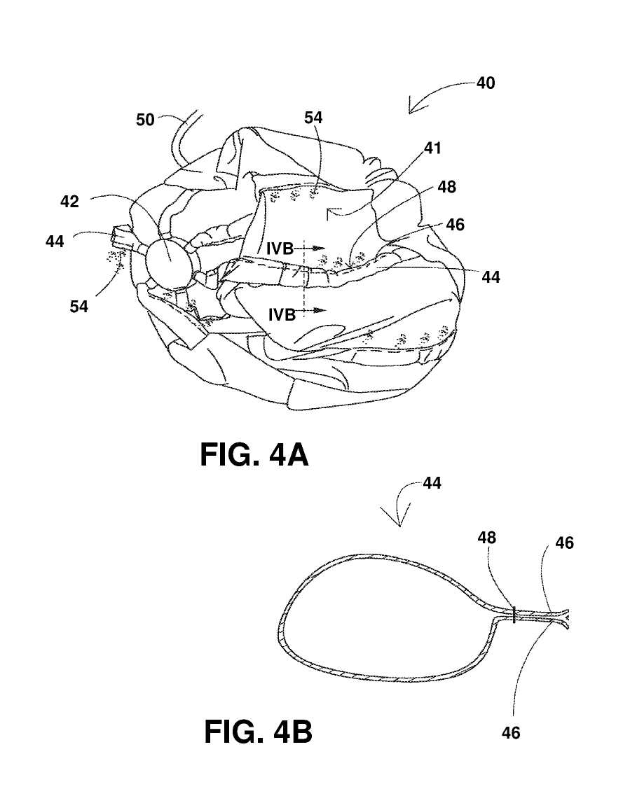

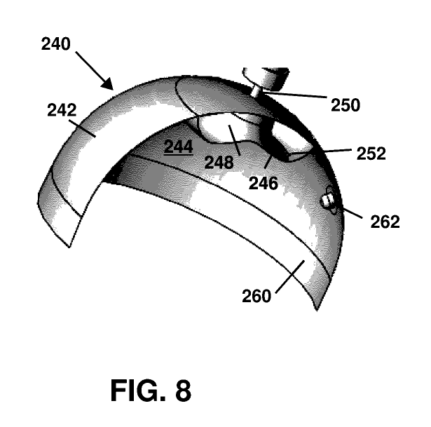

FIGS. 1 and 3 illustrate a system 10 for treating lice and nits on a head of a subject. As seen in FIG. 1, a cap 14, is adapted to be placed by a user on his or her head. The cap is typically air-tight and water-tight, and is described in further detail hereinbelow with reference to FIGS. 4A and 4B as well as with respect to FIG. 8. Cap 14 includes at least one port 16 for connecting the cap 14 to a capsule 18, either directly, as illustrated in FIG. 1, or via a first connecting tube 20, as illustrated in FIG. 3. Capsule 18 may include an active agent, as described hereinbelow with reference to FIGS. 2, 5A-5E, and 6A-6F. At an opposite end thereof, the capsule 18 is connected to a vaporizing and circulating gas source 22, either directly or via a second connecting tube 24.

In some embodiments, such as that illustrate in FIG. 1, the vaporizing and circulating gas source 22 is adapted to draw in ambient air as a vaporizing and circulating gas. For example, the vaporizing and circulating gas source 22 may comprise an electric air pump. In other embodiments, illustrated in FIG. 3, the vaporizing and circulating gas source 22 contains a vaporizing and circulating gas 26, and may include an actuating element (not shown) suitable for releasing the gas, such as a suitable handle or lever.

As explained in further detail hereinbelow, flow of the active agent included in capsule 18 from the capsule 18 to cap 14 via first connecting tube 20 is achieved by the vaporizing and circulating gas being pushed from vaporizing and circulating gas source 22 into the capsule 18. It is appreciated that the vaporizing and circulating gas may be any suitable gas, such as ambient air, pure oxygen, pure carbon dioxide, or any other suitable gas, and need not necessarily include water vapor.

Reference is now made to FIG. 2, which is a schematic illustration of a capsule 18a, which may form part of system 10 of FIG. 1, according to an embodiment of the teachings herein. As seen in FIG. 2, capsule 18a includes an immersed material 30, such as a sponge, which is immersed in a liquid active agent 32, such as acetic acid. However, the storage of the active agent in capsule 18a as shown in FIG. 2 has proven to be insufficiently effective, and to result in undesired evaporation of the active ingredient prior to use by a user or subject.

As seen in FIG. 3, capsule 18a of FIG. 2 may be used as the capsule 18 of FIG. 1.

Reference is now made to FIG. 4A, which is a schematic illustration of a cap for treating lice and nits on a head of a user according to an embodiment of the teachings herein, which cap may form part of the system of FIG. 1, and to FIG. 4B, which is a sectional illustration of an injection tube forming part of the cap of FIG. 4A, the sectional illustration taken along section lines IVB-IVB in FIG. 4A.

As seen in FIG. 4A, a cap 40, which may be used in the system of FIG. 1 in place of cap 14, is shown inside out, such that the interior surface 41 of the cap shows in the illustration. Additionally, the cap 40 is illustrated with the central part pulled and diverted toward the left side of the illustration, so as to clarify how the active agent may be distributed via the cap.

In some embodiments, the cap 40 may include a distributor 42 having a plurality of elongate injection tubes 44 extending therefrom along interior surface 41 of cap 40. Turning specifically to FIG. 4B, it is seen that injection tube 44 may be formed of a thin material, relative to its lateral section, for example fabric, which is doubled over along the length of the tube. The edge 46 of the injection tube 44 is closed, but not sealed, for example by stitches 48, such gas flowing within the interior of injection tube 44 may exit the injection tube and enter the interior of cap 40.

Returning to FIG. 4A, it is seen that distributor 42 is further connected to a connecting tube 50, similar to connecting tube 20 of FIG. 1, for example via a suitable port. Additionally, cap 40 includes a tightening band (not shown), such as an elastic band, allowing for an airtight seal around the edge of the cap 40 when worn by a subject.

In use, an active agent 54 in a gas or vapor state and at a pressure greater than that of the surrounding environment is provided to distributor 42, typically via connecting tube 50. For example, the active agent may be provided from an active agent capsule such as capsule 18 of FIG. 1, using any suitable provision means. The active agent 54 flows from distributor 42 along injection tubes 44, and is released into the air-tight interior of cap 40 via edges 46 and stitches 48 of the injection tubes 44. As such, the active agent 54 is distributed within the interior airtight cavity of the cap 40.

In some embodiments, injection tubes 44 may be placed along interior surface 41 such that treatment will target specific areas. For example, since lice and nits tend to live behind the ears and along the hairline, the injection tubes may be positioned so that the active agent 54 is released primarily in those areas, thus focusing the active areas to regions where treatment is most needed.

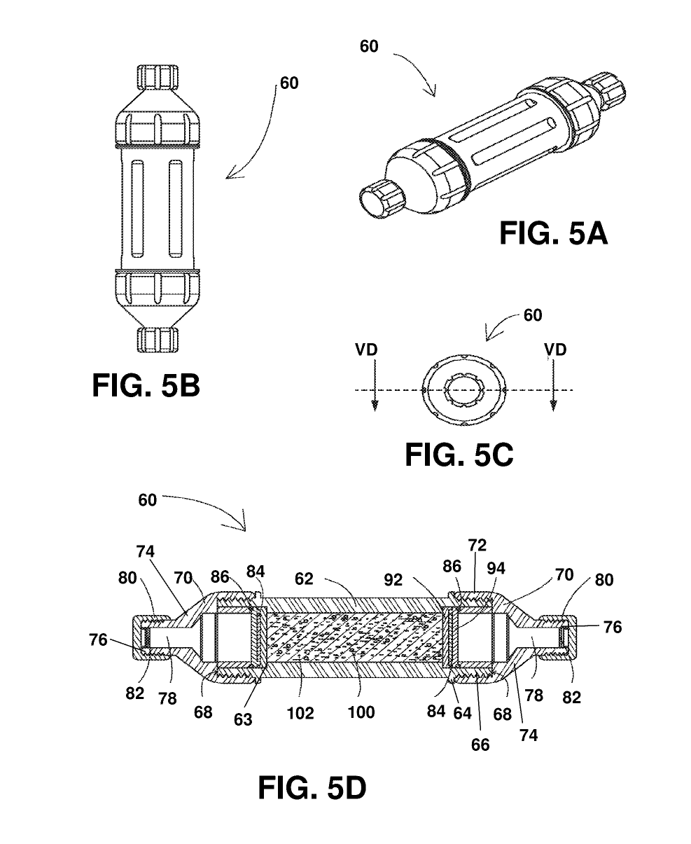

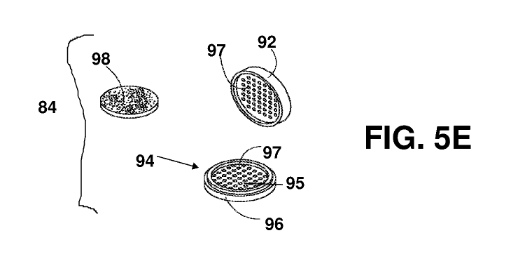

Reference is now made to FIG. 5A, which is a perspective illustration of an active agent capsule according to another embodiment of the teachings herein, which capsule may form part of the system of FIG. 1, to FIGS. 5B and 5C, which are, respectively, a side view planar illustration and a top view planar illustration of the active agent capsule of FIG. 5A, to FIG. 5D, which is a sectional view illustration of the active agent capsule of FIGS. 5A to 5C, the sectional illustration taken along section lines VD-VD in FIG. 5C, and to FIG. 5E, which is a perspective view illustration of a filter compartment and a filter forming part of the active agent capsule of FIGS. 5A to 5D.

FIGS. 5A, 5B, and 5D, illustrate an active agent capsule 60, which may be used in place of active ingredient capsule 18 of FIG. 1, in a storage state. As seen clearly in FIG. 5D, capsule includes a generally tubular main body portion 62, which in some embodiments has a circular cross section, but may have any other suitable cross section. Towards an end of body portion 62, the thickness of material of the body portion is decreased, thereby forming a step 63. At either side of main body portion there is a flange 64, followed by a connection portion 66, here illustrated as an externally threaded portion. Main body portion 62 terminates, at either end thereof, in an end wall 68.

A pair of transition portions 70, each includes a body connection portion 72 adapted to connect to connection portion 66 of the main body portion 62. In the illustrated embodiment, the body connection portion 72 is an internally threaded portion, adapted for threaded connection with the externally threaded connection portion of the main body portion. Body connection portion 72 terminates in an inwardly tapered portion 74, followed by a neck portion 76, having a fluid passageway 78 at the center thereof.

Typically, transition portions 70 have a circular cross section in all segments thereof, but they may have any suitable cross section.

Neck portion 76 is adapted to connect to a cover 80, for example by connection of external threading on neck portion 76 with internal threading on cover 80. In some embodiments, a sealing disc 82 is placed near the end of each fluid passage 78, adjacent cover 80, in order to prevent leakage of the content of capsule 60.

In some embodiments, capsule 60 may also include a pair of filters 84, sealed by a pair of O-ring seals 86. In some embodiments, the each filter includes a plurality of pores, having a pore size in the range of 0.5-0.1 mm, and preferably in the range of 0.25-0.35 mm. Turning specifically to FIG. 5E, it is seen that each filter 84 may include a filter housing compartment 90 including a first compartment portion 92, and a second compartment portion 94, between which is sandwiched a filtering component 98. The first compartment portion 92 is typically disc shaped, and sized to fit within body portion 62 such that movement of the first compartment portion towards the center of the body portion 62 is limited by step 63 thereof. The second compartment portion 94 includes a disc shaped base 95 and a circumferential cowl 96. Both the first compartment portion 92 and the disc shaped base 95 of second compartment portion 94 include holes or perforations 97 allowing access of material to and from filtering component 98.

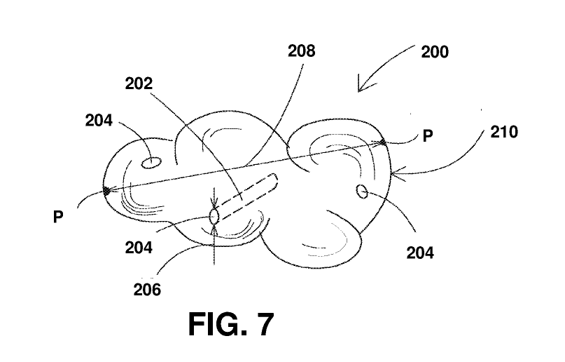

In the storage state, when active agent capsule 60 is inactive, in contains a substrate 100 having an active agent 102 in a liquid state absorbed therein or adsorbed thereto. In some embodiments, the substrate 100 includes, or is, a porous particles. For example, the substrate may be a sponge, which may be formed as a single lump, several smaller lumps, or a large number of unbound small crumb-size pieces, spherical or of another shape, including amorphous pieces. The features of the substrate are described in further detail hereinbelow with reference to FIG. 7. It will be appreciated that the substrate, and particles thereof, are designed to improve, and control timing of, vaporization of the active agent absorbed therein or adsorbed thereto.

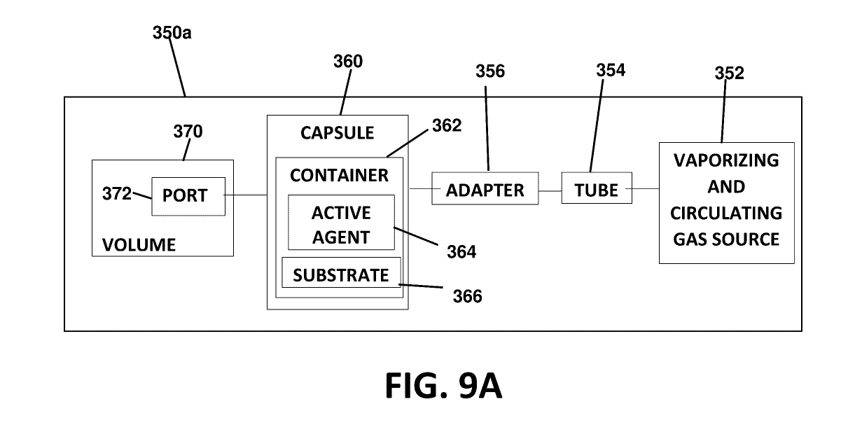

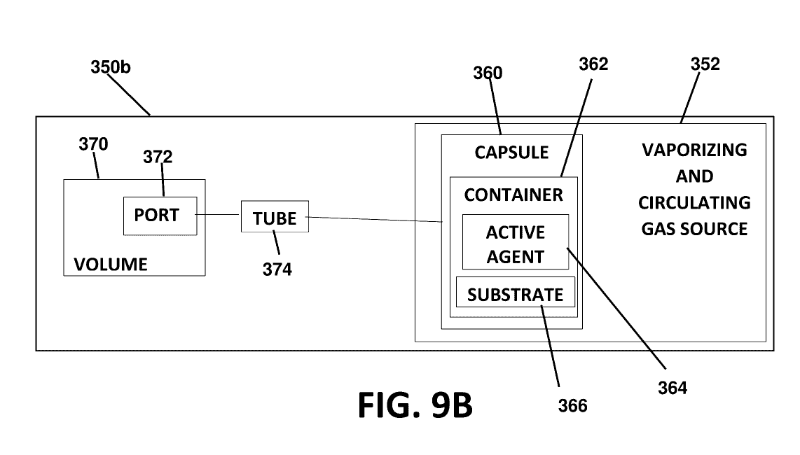

As illustrated, in the storage state, covers 80 are attached to transition portions 70, thereby sealing the capsule 60. However, it will be appreciate that in use, the covers 80 are removed, and the sealing discs 82 are broken or otherwise removed, to enable flow of the active agent out of capsule 60, as described in further detail hereinbelow with reference to FIGS. 9A and 9B. As will be explained in detail hereinbelow, in use, one of fluid passageways 78 may function as an inlet, while the other may function as an outlet.

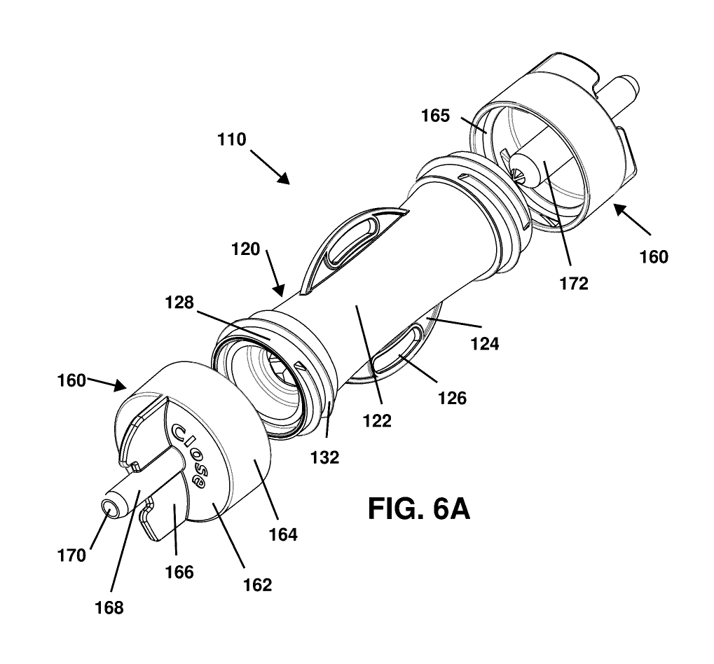

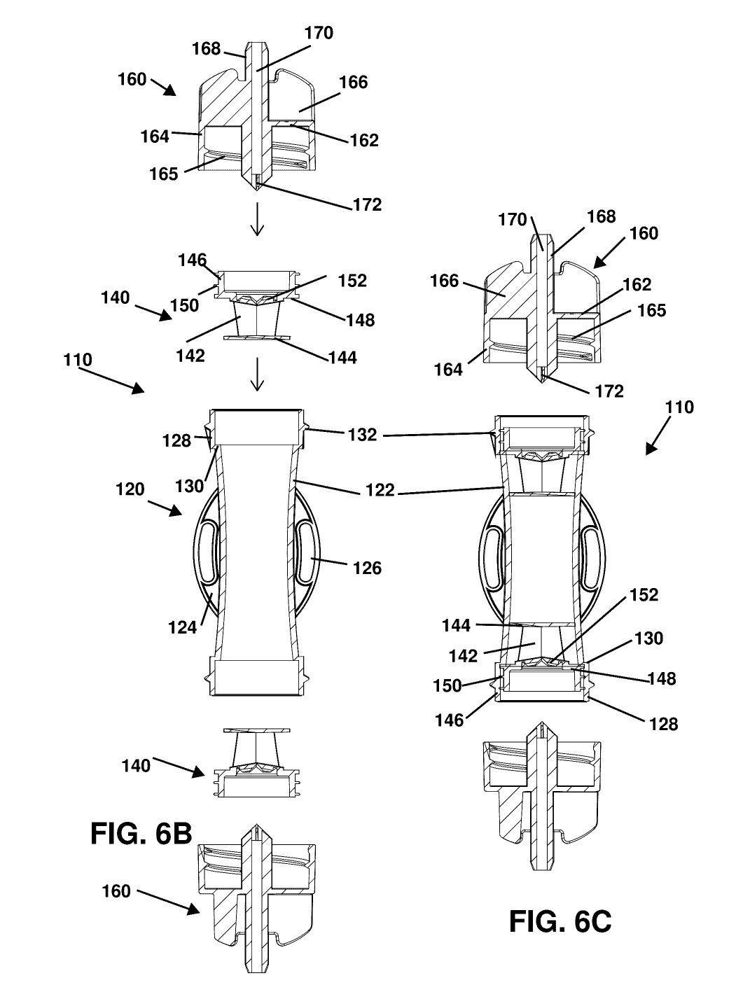

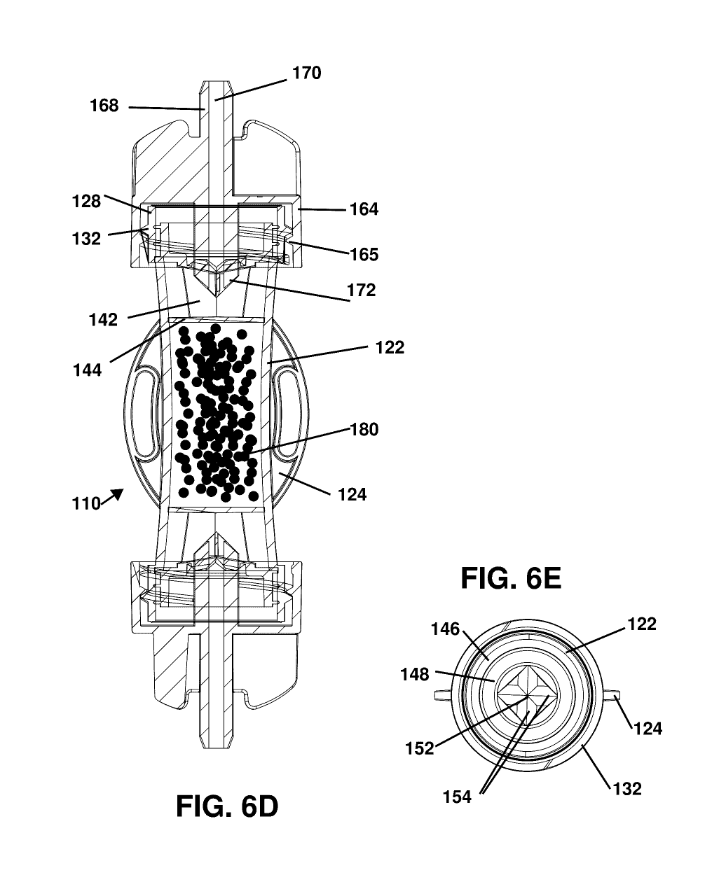

Reference is now made to FIG. 6A, which is a perspective partially exploded view illustration of an active agent capsule according to yet another embodiment of the teachings herein, which capsule may form part of the system of FIG. 1, to FIGS. 6B, 6C, and 6D, which are, respectively, a fully exploded, partially exploded, and assembled view sectional illustrations of the active agent capsule of FIG. 6A, and to FIG. 6E, which is a planar top view illustration of a filter compartment and a filter forming part of the active agent capsule of FIGS. 6A to 6D.

FIGS. 6A to 6D, illustrate an active agent capsule 110, which is similar to active agent capsule 60 of FIGS. 5A to 5D, and may be used in place of active ingredient capsule 18 of FIG. 1. As seen clearly in FIG. 6B, capsule 110 includes a body portion 120, a pair of seal portions 140, and a pair of covers 160. In some embodiments, the covers 160 are reusable and only bought once by the user, whereas the body portion 120 and seal portions 140 are intended for a single use. As such, in the storage state, the capsule 110 is typically as illustrated in FIG. 6B, where the seal portions 140 are connected to the body portion 120, but the covers 160 are detached from the body portion 120.

Body portion 120 is elongate, and includes a generally cylindrical wall 122. However, wall 122 need not be exactly cylindrical, and the cross section of the body portion 120 may vary at different locations along the length thereof. For example, in the illustrated embodiment, the cross section of the body portion is smallest at a center thereof. Extending outwardly from wall 122 are a pair of wings 124, each of which may include a generally oval cavity 126. The wings 124 are designed to allow the user to better grip the body portion 120 when connecting it to covers 160, as described hereinbelow. However, in some embodiments, the wings 124 may be obviated.

At either end of wall 122 there is a cowl portion 128, having a cross section which is slightly greater than that of wall portion 122, such that a step 130 is formed between the wall 122 and cowl portion 128. Cowl portions 128 are designed for connection to covers 160, for example by threading 132 disposed on the exterior surface of cowl portions 128.

Each of seal portions 140 includes a hollow body portion 142, which in some embodiments may have a square or rectangular cross section. In some embodiments, body portion 142 may comprise a truncated square pyramid. At one end thereof, body portion 142 terminates at a disc shaped surface 144, which is typically adapted for fluid flow, for example by including a central opening, or a plurality of smaller distributed openings, cavities, or perforations. In some embodiments, surface 144 may be, or may form part of, a filter, substantially as described hereinabove with respect to FIG. 5E. Extending at the opposite end of body portion 142 is a cowl portion 146. Cowl portion 146 is generally cylindrical, and has a greater cross section than that of body portion 142, such that a step 148 is formed between body portion 142 and cowl portion 146. A plurality of circumferential protrusions 150 are formed on the exterior of cowl portion 146, which are adapted to create a seal between cowl portion 146 and body portion 120 as described hereinbelow. A seal 152 is disposed at the connection between body portion 142 and cowl portion 146, and continues step 148 to provide an airtight and watertight seal between the body portion and the cowl portion.

In some embodiments, such as the embodiment illustrated in FIG. 6E, seal 152 is formed of multiple leaves 154, intersecting at a central point, thus forming a shape similar to that of a flower or of a sliced pizza pie. The intersection at a central point is significant to the unsealing of the capsule 110, as will be described in further detail hereinbelow.

Each of covers 160 includes a generally cylindrical base 162, from one side of which extends a generally cylindrical connector portion 164, adapted to connect to cowl portion 128 of main body 120. In some embodiments, connector portion 164 is incudes internal threading 165 to enable threaded connection with external threading of cowl portion 128, although any other suitable connection mechanism is considered within the scope of the present invention. Extending from the opposite side of cylindrical base 162 is a pair of flaps 166, adapted to provide a finger-hold for rotation of cover 160. Extending along the center of cover 160, between flaps 166, through base 162 and in the center of connector portion 164, is a hollow tube 168 defining therein a fluid flow channel 170 and terminating at an end thereof in a spike 172.

As seen clearly in FIG. 6D, the hollow interior of main body 120 contains a substrate 180 having an active agent in a liquid state absorbed therein or adsorbed thereto. In some embodiments, the substrate 180 includes, or is, a porous particles. For example, the substrate may be a sponge, which may be formed as a single lump, several smaller lumps, or a large number of unbound small crumb-size pieces, spherical or of another shape, including amorphous pieces. The features of the substrate are described in further detail hereinbelow with reference to FIG. 7. It will be appreciated that the substrate, and particles thereof, are designed to improve, and control timing of, vaporization of the active agent absorbed therein or adsorbed thereto.

Turning to FIGS. 6C and 6D, it is seen that in a first construction stage, which is typically performed by a manufacturer, seal portions 140 are inserted into main body 120 from either side thereof The seal portions lodge in main body 120, and are prevented from motion towards the center of the main body, by step 148 of the seal portion engaging step 130 of the main body. The protrusions 150 of cowl portion 146 engage an inner surface of cowl portion 128 of the main body and create a seal therewith.

In some embodiments, connector portions 164 of covers 160 may be partially connected to cowl portions 128 of main body 120 by the manufacturer, for example by screwing threads 132 and 165 to one another. In some embodiments the presence of covers 160 prevents seal portions 140 from moving out of main body 120.

It is a particular feature of the present invention that in the storage state, prior to use of the capsule 110, the covers 160 are disconnected from body portion 120, or are only partially screwed onto body portion 120, such that spike 172 does not break seal 152, and the substrate 180 and active agent 182 remain sealed within the hollow of body portion 120.

When preparing for use, the user may connect covers 160 to main body 120, or may twist covers 160 to fully tighten screwing thereof, for example by holding flaps 166, thereby causing spike 172 to be pushed further towards seal portion 140 and to rupture seal 152, for example at the point at which leaves 154 intersect. Such puncturing of seal 152 enables fluid flow into and out of the interior hollow of body portion 120, via channels 170 of the covers 160. As will be explained in detail hereinbelow, in use, one of channels 170 may function as an inlet, while the other may function as an outlet.

In some embodiments, capsule 60 and/or capsule 110 is designed for single time use. For example, in some embodiments, the quantity of active agent stored within capsule 60 or 110 is sufficient for a single use, but not more than that. As another example, covers 80 and/or sealing discs 82 of capsule 60 may be destroyed by opening or removal thereof, such that the capsule can no longer be closed, resulting in spontaneous evaporation of any residual active agent stored therein after treatment. Similarly, puncturing of seal 152 by spike 172 ensures that the active agent within capsule 110 can no longer be sealed, resulting in spontaneous evaporation of any residual active agent stored therein after treatment.

In the illustrated embodiment, in which the goal is to treat a lice infestation on the head of a human subject, the active agent 102 or may be any liquid active agent suitable for treating the lice infestation. One particularly effective active agent 102 or 182 for this specific purpose is acetic acid (CH₃COOH).

However, it will be appreciated by persons of skill in the art that the capsule 60 or 110, and the vaporization system 10 with which it may be used, may be used for other applications as well. For example, the system may be used for treating furniture or other inanimate objects against vermin. In such embodiments, instead of using an airtight cap, an airtight space may be formed around the object, and a suitable vermicide may be used as the active agent stored in the capsule. Such embodiments are considered to be within the scope of the present invention.

Reference is now made to FIG. 7, which is a schematic illustration of a porous particle 200 which may be used as a substrate in an active ingredient capsule, such as the substrate 100 of FIGS. 5A to 5E or the substrate 180 of FIGS. 6A to 6E, according to an embodiment of the teachings herein.

The porous particle 200 is typically an inert component, miniscule relative to the dimensions of the capsule 18 (FIG. 1), 60 (FIGS. 5A-5D), or 110 (FIG. 6A-6D), having pores 202. Each pore 202 has at least one pore opening 204, having an opening diameter 206 in the order of magnitude of single to several tens of microns. The porous particle 200 has a porous particle size 208 in the order of magnitude of several tens to several hundreds of microns. The porous particle size 208 is defined as the largest distance between two surface points P on an outer surface 210 of porous particle 200. The porous particle 200 may be amorphous or of a defined spatial shape, such as a cylinder, a sphere, etc., and may be composed of materials such as thermoplastic polymers, glass, etc.

One particularly suitable type of porous particle 200 is a Ceramic Filtering Ball commercially available from Pingxiang BaiSheng Chemical Packing Company, Ltd. of Pingxiang City, Jinagxi Province, P.R.China, having a size in the range of 1-2 mm, a pore size in the range of 110-1000 microns, and water adsorption of 12-15%.

Reference is now made to FIG. 8, which is a schematic illustration of another cap for treating lice and nits on a head of a user according to another embodiment of the teachings herein, which cap may form part of the system of FIG. 1.

As seen in FIG. 8, a cap 240, which may be used in the system of FIG. 1 in place of cap 14, includes a main body 242 defining a main hollow 244. At a top portion of main body 242, which is adapted to be placed on the crown of a user's head, the cap includes a ring shaped lining 246 attached at an end thereof to main body 242 and forming a hollow 248 between the main body and the lining The hollow 248 is fluidly connected to an input port 250 enabling fluid flow into the cap. A plurality of openings 252 are distributed about lining 246 so that hollow 248 is in fluid flow communication with the main hollow 244. In some embodiments, lining 246 prevents any active ingredient reaching input port 250 in liquid form from being released into the hollow 244 and onto the user's head, thereby meeting regulatory requirements.

Cap 240 terminates in a sealing element 260, such as an elastic band, adapted to form an air-tight and fluid-tight seal around the user's head, when the cap is worn. The sealing element 260 may be any suitable sealing element, as known in the art. In some embodiments, cap 240 further includes a pressure release valve 262 adapted for release of pressure from within the cap, in use.

Reference is now made to FIGS. 9A and 9B, which are schematic block diagrams of two embodiments of a system for treating an object with gas or vapor according to additional embodiments of the teachings herein.

The systems 350a and 350b of respective FIGS. 9A and 9B are similar to the system 10 of FIG. 3, with a few important differences. The systems 350a and 350b each include a vaporizing and circulating gas source 352 adapted to provide a vaporizing and circulating gas, either from the ambient environment of the system or from a dedicated vaporizing and circulating gas storage (not shown). The vaporizing and circulating gas source 352 is fluidly attached to a capsule 360.

In some embodiments, such as the embodiment of FIG. 9A, the gas source 352 is attached to capsule 360 via a tube 354 and a suitable adapter 356. In other embodiments, such as the embodiment of FIG. 9B, the capsule 360 is housed within the housing of gas source 352, such that it is in fluid flow communication therewith.

The capsule 360 includes therein a container 362, containing a liquid active agent 364 absorbed in or adsorbed to a substrate 366. For example, capsule 360 may be similar to capsule 60 of FIGS. 5A-5D, with the container 362 being equivalent to main body 62 thereof, the substrate 366 being equivalent to substrate 100, and the active agent 364 being equivalent to active agent 102. As another example, capsule 360 may be similar to capsule 110 of FIGS. 6A-6D, with the container 362 being equivalent to main body 120 thereof, the substrate 366 being equivalent to substrate 180.

In the embodiment of FIG. 9A, adapter 358 is adapted, in use, to interact with capsule 360, so as to break or otherwise open a seal thereof thereby allowing fluid flow into and out of container 362. For example, adapter 358 may be equivalent to spikes 172, which puncture the seal 152 thereby providing fluid flow communication with the interior of body portion 120.

The capsule 360 is fluidly attached, at another end thereof, to an airtight treatment volume 370 via a port 372. Volume 370 may, for example, be a cap to be placed on a subject's head for the treatment of lice and nits, or an airtight wrapper placed around an object for the treatment of vermin. In some embodiments, the capsule may be directly connected to the treatment volume, possibly via an adapter similar to adapter 356 described above, as illustrated in FIG. 9A. In other embodiments, fluid flow connection between capsule 360 and volume 370 may be via a connecting tube 374, as illustrated in FIG. 9B.

It will be appreciated by people skilled in the art that the fluid flow connections between gas source 352 and capsule 360, and between capsule 360 and volume 370, may be achieved using any suitable means. As such, these connections may include one or more connecting tubes. Alternately, these connections may be direct, requiring no connecting tubes, depending on the structure suitable for each specific application and active agent.

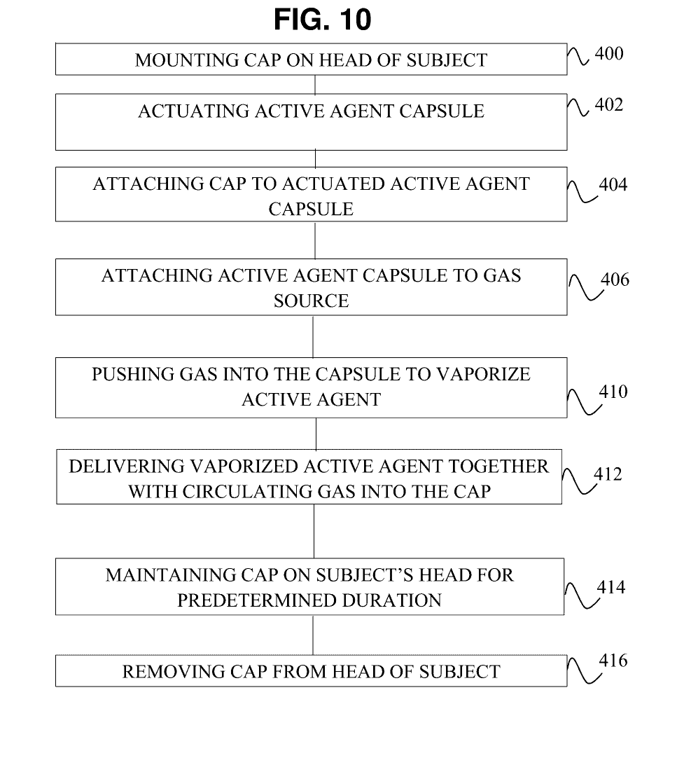

Reference is now made to FIG. 10, which is a flow chart of a method for treating lice and nits on a head of a subject, according to an embodiment of the teachings herein.

As seen in FIG. 10, at step 400 a cap is mounted on the head of a subject to be treated, so as to form an airtight volume around the head of the subject. The cap may be any suitable cap, for example as described hereinabove with reference to FIGS. 4A and 4B.

In some embodiments, in which an active agent capsule is sealed until use thereof, the active agent capsule is actuated, or unsealed, at step 402. For example, in the embodiment of FIGS. 6A-6E, the actuation or unsealing of the capsule involves rotating covers 160 sufficient for spikes 172 to perforate sealing mesh 152, thereby to enable fluid flow of the active agent 182, adsorbed onto substrate 180 within the body portion of the capsule, into channels 170 of covers 160.

At step 404, the cap is attached to the actuated active agent capsule, such as active agent capsule 60 of FIGS. 5A-5D or active agent capsule 110 of FIGS. 6A-6E. As described hereinabove, in some embodiment the attachment between the cap and the capsule is via a suitable connecting element, such as a connecting tube. As discussed hereinabove, the capsule includes an active agent, which, in some embodiments, may be in liquid form and may be absorbed in or adsorbed to a substrate or other immersed material.

At step 406, the active agent capsule is connected to a gas source, such as vaporizing and circulating gas sources 22, 222, and 352 of FIGS. 1, 8, and 9, respectively. As discussed hereinabove, the connection may be direct or may be via one or more connecting elements, such as connecting tubes.

It will be appreciated that the connection of the active agent capsule to the cap and to the gas source, and mounting of the cap on the subject's head, may be carried out in any suitable order. For example, the capsule may be connected to the gas source prior to being connected to the cap, and/or the cap may be mounted on the subject's head only once it has been connected to the capsule.

Once the cap, capsule, and gas source are connected to one another, the system is ready for use. As such, at step 410 a predetermined volume of vaporizing and circulating gas is pushed into the capsule to surround the substrate and active agent therein, and to trigger vaporization of the active agent. A suitable volume of the vaporized active agent is delivered, together with the vaporizing and circulating gas, into the cap, at step 412.

The cap is maintained on the subject's head for a predetermined duration, at step 414. Subsequently, at step 416, the cap is removed from the subject's head, and the treatment is complete. In some embodiments, for example when treating the subject's head for lice using the concentration and volume of acetic acid vapor described hereinabove, the cap may be maintained on the subject's head for a duration in the range of 5 minutes to 15 minutes. In embodiments in which the treatment is used for another object, the treatment volume may be maintained surrounding the object for a duration in the range of 5 minutes to 4 hours.

As discussed hereinabove, the system and method described herein are suitable for treatment of many objects by provision of a vaporized active agent to an airtight volume surrounding the object. It will be appreciated that, in embodiments which do not include providing vaporized active agent to the head of a subject, but rather to another body part of the subject or to another object, the cap described in the method may be replaced by any suitable airtight cover wrapping the body part or object to be treated for forming an airtight volume around the body part or object.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the scope of the appended claims.

Citation or identification of any reference in this application shall not be construed as an admission that such reference is available as prior art to the invention.

Section headings are used herein to ease understanding of the specification and should not be construed as necessarily limiting.

1. A capsule for storage and delivery of an active agent, the capsule comprising:
a container defining a storage volume, said storage volume containing a substrate having a volume of liquid active agent adsorbed onto said substrate or absorbed in said substrate;
at least one fluid flow passage into and out of said storage volume; and
at least one seal sealing said at least one fluid flow passage and preventing flow of fluid therefrom,
wherein said capsule is sealed to fluid flow out of said capsule.

2. The capsule of claim 1, wherein said container is formed of a fluid impassable material.

3. The capsule of claim 1, wherein said substrate comprises a plurality of unbound miniscule particles.

4. The capsule of claim 1, further comprising at least one cover adapted to be disposed about said at least one seal, wherein said at least one cover comprises:
a puncturing element adapted to puncture said seal; and
at least one channel,
wherein, in an actuated state of said capsule, puncturing element punctures said seal such that said at least one channel is in fluid flow communication with said at least one fluid flow passage, and fluid can flow into and out of said storage volume via said at least one fluid flow passage and via said at least one channel.

5. The capsule of claim 4, wherein, in said actuated state of said capsule, said cover is positioned axially closer to said storage volume than in a storage state of said capsule, thereby causing said puncturing element to puncture said seal.

6. The capsule of claim 4, wherein:
said capsule comprises two said seals sealing said storage volume;
said capsule comprises two said covers, each including a said puncturing element and a said channel, each puncturing element being aligned with a corresponding one of said two seals; and
in said actuated state, each of said puncturing elements punctures a said seal corresponding thereto, thereby to allow flow of fluid in said corresponding channel.

7. The capsule of claim 6, wherein, in said actuated state, a first of said two channels functions as an inlet and a second of said two channels functions as an outlet.

8. The capsule of claim 6, wherein said two seals and said two corresponding covers are disposed axially on opposite sides of said storage volume.

9. The capsule of claim 1, further comprising at least one filter disposed between said storage volume and said fluid flow passage, and enabling filtering of fluid flowing through said fluid flow passage following removal or breakage of said seal.

10. The capsule of claim 9, wherein said at least one filter prevents passage of said substrate and allows passage of a vapor of said liquid active agent.

11. A system for evaporating and circulating an active agent disposed in a capsule, the system comprising:
the capsule of claim 4;
a vaporizing and circulating gas source in fluid flow communication with said storage volume via said at least one channel,
said gas source adapted to emit a vaporizing a circulating gas into said storage volume thereby to increase pressure within said storage volume, said increase in pressure causing vaporization of said active agent and release thereof from said substrate.

12. The system of claim 11, wherein said vaporizing and circulating gas is selected from the group consisting of: pure oxygen, pure carbon dioxide, and ambient air.

13. The system of claim 11, wherein:
said capsule comprises a first and a second said seal sealing said storage volume;
said capsule comprises a first and a second said cover, each including a said puncturing element and a said channel, each puncturing element being aligned with a corresponding one of said first and second seals;
in said actuated state, each of said puncturing elements punctures a said seal corresponding thereto, thereby to allow flow of fluid out of said storage volume via said corresponding channel, the fluid including a mixture of a vapor of said active agent and said vaporizing and circulating gas; and
said gas source is in fluid flow communication with a said channel of said first cover, said channel of said first cover functioning as an inlet into said storage volume.

14. The system of claim 13, further comprising:
an object to be treated with said active agent; and
a fluid-tight enclosure enclosing said object and defining an internal treatment volume, said internal treatment volume being in fluid flow communication with said channel of said second cover,
wherein said gas flows from said gas source into said storage volume, increases pressure within said storage volume thereby vaporizing said active agent, and circulates vapor of said active agent, via said channel of said second cover, into said fluid tight enclosure, for treatment of said object.

15. The system of claim 14, wherein said object is a head of a subject.

16. The capsule of claim 1, wherein said active agent is acetic acid.
